(19) United States
(12) Patent Application Publication
(10) Pub. No.: US 2007/0244097 A1
(43) Pub. Date: Oct. 18, 2007

(54) Nicotinic AcetylcholineReceptor Ligands

(75) Inventors: Glen Ernst, Wilmington, DE (US); William Frietze, Wilmington, DE (US); Robert Jacobs, Research Triangle Park, NC (US); Eifion Phillips, Boothwyn, PA (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 10/583,585

(22) PCT Filed: Dec. 20, 2004
(86) PCT No.: PCT/SE04/01942

§ 371 Date: Apr. 10, 2007

Related U.S. Application Data
(60) Provisional application No. 60/531,644, filed on Dec. 22, 2003.

(51) Int. Cl. A61K 31/55; A61P 25/00; C07D 243/08; C07D 487/08

(52) U.S. Cl. 514/221; 540/575

(57) ABSTRACT

Acetylcholine receptor ligands of formula I wherein D, E and G are as described in the specification, diastereoisomers, enantiomers, pharmaceutically-acceptable salts, methods of making, pharmaceutical compositions containing and methods for using the same.

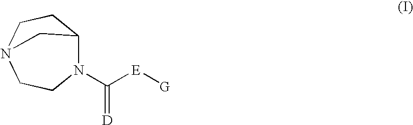

### FIELD OF THE INVENTION

This invention relates to diazabicyclo-octyl amides or pharmaceutically-acceptable salts thereof, processes for preparing them, pharmaceutical compositions containing them and their use in therapy. The invention also relates to compounds that are ligands for nicotinic acetylcholine receptors (nAChRs).

### BACKGROUND OF THE INVENTION

The use of compounds which bind nicotinic acetylcholine receptors in the treatment of a range of disorders involving reduced cholinergic function such as Alzheimer's disease, cognitive or attention disorders, anxiety, depression, smoking cessation, neuroprotection, schizophrenia, analgesia, Tourette's syndrome, and Parkinson's disease has been discussed in McDonald et al. (1995) “Nicotinic Acetylcholine Receptors: Molecular Biology, Chemistry and Pharmacology”, Chapter 5 in Annual Reports in Medicinal Chemistry, vol. 30, pp. 41-50, Academic Press Inc., San Diego, Calif.; and in Williams et al. (1994) “Neuronal Nicotinic Acetylcholine Receptors,” Drug News & Perspectives, vol. 7, pp. 205-223.

### DESCRIPTION OF THE INVENTION

This invention concerns nicotinic acetylcholine receptor-active compounds according to formula I: 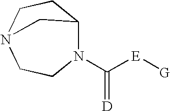
wherein:

D is selected from oxygen, sulfur or N(R¹)₂;

E is selected from —C(R¹)₂—C(R¹)₂—, —CR¹═CR¹—, —C≡C— and —C(R¹)₂—O—

G is selected from a 5- or 6-membered aromatic or heteroaromatic ring having 0, 1 or 2 nitrogen atoms, 0 or 1 oxygen atoms, and 0 or 1 sulfur atoms, or selected from an 8-, 9- or 10-membered fused aromatic or heteroaromatic ring system having 0, 1, 2 or 3 nitrogen atoms, 0 or 1 oxygen atoms, and 0 or 1 sulfur atoms;

where G is unsubstitnted or has 1, 2 or 3 substituents selected from —C₁-C₆alkyl, —C₂-C₆alkenyl, —C₂-C₆alkynyl, halogen, —CN, —NO₂, —CF₃, —S(O)ₙR³, —NR²R³, —CH₂NR²R³, —OR³, —CH₂OR³ or —CO₂R⁴;

R¹, R² and R³ are independently selected at each occurrence from hydrogen, halogen, —C₁-C₄alkyl, aryl, heteroaryl, —C(O)R⁴, —C(O)NHR⁴, —CO₂R⁴ or —SO₂R⁴, or

R² and R³ in combination is —(CH₂)ⱼG(CH₂)ₖ— wherein G is oxygen, sulfur, NR⁴, or a bond;

j is 2, 3 or 4;

k is 0, 1 or 2;

n is 0, 1 or 2, and

R⁴ is independently selected at each occurrence from hydrogen, —C₁-C₄alkyl, aryl, or heteroaryl.

The invention also encompasses stereoisomers, enantiomers, in vivo-hydrolysable precursors and pharmaceutically-acceptable salts of compounds of formula I, pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

Compound of the invention are those according to formula I: 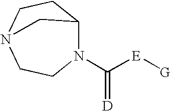
wherein:

D is selected from oxygen, sulfur or N(R¹)₂;

E is selected from —C(R¹)₂—C(R¹)₂—, —CR¹═CR¹—, —C≡C— or —C(R¹)₂—O—;

G is selected from a 5- or 6-membered aromatic or heteroaromatic ring having 0, 1 or 2 nitrogen atoms, 0 or 1 oxygen atoms, and 0 or 1 sulfur atoms, or selected from an 8-, 9- or 10-membered fused aromatic or heteroaromatic ring system having 0, 1, 2 or 3 nitrogen atoms, 0 or 1 oxygen atoms, and 0 or 1 sulfur atoms;

where G is unsubstituted or has 1, 2 or 3 substituents selected from —C₁-C₆alkyl, —C₂-C₆alkenyl, —C₂-C₆alkynyl, halogen, —CN, —NO₂, —CF₃, —S(O)ₙR³, —NR²R³, —CH₂NR²R³, —OR³, —CH₂OR³ or —CO₂R⁴;

R¹, R² and R³ are independently selected at each occurrence from hydrogen, halogen, —C₁-C₄alkyl, aryl, heteroaryl, —C(O)R⁴, —C(O)NHR⁴, —CO₂R⁴ or —SO₂R⁴, or

R² and R³ in combination is —(CH₂)ⱼG(CH₂)ₖ— wherein G is oxygen, sulfur, NR⁴, or a bond;

j is 2, 3 or 4;

k is 0, 1 or 2;

n is 0, 1 or 2, and

R⁴ is independently selected at each occurrence from hydrogen, —C₁-C₄alkyl, aryl, or heteroaryl,

and stereoisomers, enantiomers, in vivo-hydrolysable precursors and pharmaceutically-acceptable salts thereof.

Particular compounds are those of formula I wherein:

D is oxygen;

E is selected from —C(R¹)₂—C(R¹)₂—, —CR¹═CR¹—, —C≡C— or —C(R¹)₂—O—;

G is phenyl;

where G is unsubstituted or has 1, 2 or 3 substituents selected from —C₁-C₆alkyl and

R¹ is independently selected at each occurrence from hydrogen or halogen.

More particular compounds are those of formula I wherein:

D is oxygen;

E is selected from —C(R¹)₂—C(R¹)₂—, —CR¹═CR¹—, —C≡C— or —C(R¹)₂—O—;

G is phenyl;

where G is unsubstituted or has methyl substituent and

R¹ is independently selected at each occurrence from hydrogen or fluoro.

Particular compounds of the invention are those described herein and pharmaceutically-acceptable salts thereof.

In a further aspect the invention encompasses compounds according to formula I wherein one or more of the atoms is a radioisotope of the same element. In a particular form of this aspect of the invention the compound of formula I is labeled with tritium. Such radio-labeled compounds are synthesized either by incorporating radio-labeled starting materials or, in the case of tritium, exchange of hydrogen for tritium by known methods. Known methods

Another embodiment of this aspect of the invention is a method of treatment or prophylaxis of jetlag, nicotine addiction, craving, pain, and for ulcerative colitis, which comprises administering a therapeutically effective amount of a compound of the invention.

Yet another embodiment of this aspect of the invention is a method for inducing the cessation of smoking which comprises administering an effective amount of a compound of the invention.

Another embodiment of this aspect of the invention is a pharmaceutical composition comprising a compound of the invention and a pharmaceutically-acceptable diluent, lubricant or carrier.

A further aspect of the invention relates to a pharmaceutical composition useful for treating or preventing a condition or disorder mentioned herein arising from dysfunction of nicotinic acetylcholine receptor neurotransmission in a mammal, preferably a human, comprising an amount of a compound of formula I, an enantiomer thereof or a pharmaceutically-acceptable salt thereof, effective in treating or preventing such disorder or condition, and pharmaceutically-acceptable additives carrier.

Another embodiment of this aspect of the invention relates to use of a pharmaceutical composition of the invention for the treatment, amelioration or prophylaxis of human diseases or conditions in which activation of the α7 nicotinic receptor is beneficial.

Another embodiment of this aspect of the invention is the use of the pharmaceutical composition of the invention for the treatment or prophylaxis of neurological disorders, psychotic disorders or intellectual impairment disorders.

Another embodiment of this aspect of the invention is the use of the pharmaceutical composition of the invention for the treatment or prophylaxis of Alzheimer's disease, learning deficit, cognition deficit, attention deficit, memory loss, Attention Deficit Hyperactivity Disorder, anxiety, schizophrenia, or mania or manic depression, Parkinson's disease, Huntington's disease, Tourette's syndrome, neurodegenerative disorders in which there is loss of cholinergic synapse, jetlag, cessation of smoking, nicotine addiction including that resulting from exposure to products containing nicotine, craving, pain, and for ulcerative colitis.

A further aspect of the invention is the use of a compound according to the invention, an enantiomer thereof or a pharmaceutically-acceptable salt thereof, in the manufacture of a medicament for the treatment or prophylaxis of the diseases or conditions mentioned herein. include (1) electrophilic halogenation, followed by reduction of the halogen in the presence of a tritium source, for example, by hydrogenation with tritium gas in the presence of a palladium catalyst, or (2) exchange of hydrogen for tritium performed in the presence of tritium gas and a suitable organometallic (e.g. palladium) catalyst.

Compounds of the invention labeled with tritium are useful for the discovery of novel medicinal compounds which bind to and modulate the activity, by agonism, partial agonism, or antagonism, of the α7 nicotinic acetylcholine receptor. Such tritium-labeled compounds may be used in assays that measure the displacement of a such compounds to assess the binding of ligand that bind to α7 nicotinic acetylcholine receptors.

In another aspect the invention relates to compounds according to formula I and their use in therapy and to compositions containing them.

In another aspect the invention encompasses the use of compounds according to formula I for the therapy of diseases mediated through the action of nicotinic acetylcholine receptors. A more particular aspect of the invention relates to the use of compounds of formula I for the therapy of diseases mediated through the action of α7 nicotinic acetylcholine receptors.

Another aspect of the invention encompasses a method of treatment or prophylaxis of diseases or conditions in which activation of the α7 nicotinic receptor is beneficial which method comprises administering a therapeutically-effective amount of a compound of the invention to a subject suffering from said disease or condition.

One embodiment of this aspect of the invention is a method of treatment or prophylaxis, wherein the disorder is anxiety, schizophrenia, mania or manic depression.

Another embodiment of this aspect of the invention is a method of treatment or prophylaxis of neurological disorders, psychotic disorders or intellectual impairment disorders, which comprises administering a therapeutically effective amount of a compound of the invention.

Another embodiment of this aspect of the invention is a method of treatment or prophylaxis, wherein the disorder is Alzheimer's disease, learning deficit, cognition deficit, attention deficit, memory loss, or Attention Deficit Hyperactivity Disorder.

Another embodiment of this aspect of the invention is a method of treatment or prophylaxis, wherein the disorder is Parkinson's disease, Huntington's disease, Tourette's syndrome, or neurodegenerative disorders in which there is loss of cholinergic synapses.

Another embodiment of this aspect of the invention is the use of a compound of the invention in the manufacture of a medicament for the treatment or prophylaxis of human diseases or conditions in which activation of the α7 nicotinic receptor is beneficial.

Another embodiment of this aspect of the invention is the use of a compound of the invention in the manufacture of a medicament for the treatment or prophylaxis of neurological disorders, psychotic disorders or intellectual impairment disorders.

Another embodiment of this aspect of the invention is the use of a compound of the invention in the manufacture of a medicament for treatment or prophylaxis of Alzheimer's disease, learning deficit, cognition deficit, attention deficit, memory loss or Attention Deficit Hyperactivity Disorder.

Another embodiment of this aspect of the invention is the use of a compound of the invention in the manufacture of a medicament for treatment or prophylaxis of anxiety, Schizophrenia, or mania or manic depression.

Another embodiment of this aspect of the invention is the use of a compound of the invention in the manufacture of a medicament for treatment or prophylaxis of Parkinson's disease, Huntington's disease, Tourette's syndrome, or neurodegenerative disorders in which there is loss of cholinergic synapses.

Another embodiment of this aspect of the invention is the use of a compound as described above in the manufacture of a medicament for the treatment or prophylaxis of jetlag, pain, or ulcerative colitis.

Another aspect of the invention relates to the use of a compound of the invention in the manufacture of a medicament for facilitating the cessation of smoking or the treatment of nicotine addiction or craving including that resulting from exposure to products containing nicotine.

For the uses, methods, medicaments and pharmaceutical compositions mentioned herein the amount of compound used and the dosage administered will, of course, vary with the compound employed, the mode of administration and the treatment desired. However, in general, satisfactory results are obtained when the compounds of the invention are administered at a daily dosage of from about 0.1 mg to about 20 mg/kg of animal body weight. Such doses may be given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carriers, lubricants and diluents.

The compounds of formula I, an enantiomer thereof, and pharmaceutically-acceptable salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically-acceptable diluent, lubricant or carrier.

Examples of diluents, lubricants and carriers are

for tablets and dragees: lactose, starch, talc, stearic acid;
for capsules: tartaric acid or lactose;
for injectable solutions: water, alcohols, glycerin, vegetable oils;
for suppositories: natural or hardened oils or waxes.

There is also provided a process for the preparation of such a pharmaceutical composition which process comprises mixing the ingredients.

Compounds according to the invention are agonists of nicotinic acetylcholine receptors. While not being limited by theory, it is believed that agonists of the α7 nicotinic acetylcholine receptor (nAChR) subtype are useful in the treatment or prophylaxis of neurological disorders, psychotic disorders and intellectual impairment disorders, and to have advantages over compounds which are or are also agonists of the α4 nAChR subtype. Therefore, compounds which are selective for the α7 nAChR subtype are preferred. The compounds of the invention are indicated as pharmaceuticals, in particular in the treatment or prophylaxis of neurological disorders, psychotic disorders and intellectual impairment disorders. Examples of psychotic disorders include schizophrenia, mania and manic depression, and anxiety. Examples of intellectual impairment disorders include Alzheimer's disease, learning deficit, cognition deficit, attention deficit, memory loss, and Attention Deficit Hyperactivity Disorder. The compounds of the invention may also be useful as analgesics in the treatment of pain, chronic pain, and in the treatment or prophylaxis of Parkinson's disease, Huntington's disease, Tourette's syndrome, and neurodegenerative disorders in which there is loss of cholinergic synapses.

Compounds of the invention may further useful for the treatment or prophylaxis of jetlag, for use in inducing the cessation of smoking, craving, and for the treatment or prophylaxis of nicotine addiction including that resulting from exposure to products containing nicotine.

It is also believed that compounds according to the invention are useful in the treatment and prophylaxis of ulcerative colitis.

The compounds of the invention have the advantage that they may be less toxic, be more efficacious, be longer acting, have a broader range of activity, be more potent, produce fewer side effects, are more easily absorbed or have other useful pharmacological properties.

The compounds of formula I exist in tautomeric or enantiomeric forms, all of which are included within the scope of the invention. The various optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

As used herein, unless otherwise indicated, “C₁₋₄alkyl” includes but is not limited to methyl, ethyl, n-propyl, n-butyl, i-propyl, i-butyl, t-butyl, s-butyl moieties, whether alone or part of another group, C₁₋₄alkyl groups may be straight-chained or branched, and C₃₋₄ alkyl groups include the cyclic alkyl moieties cyclopropyl and cyclobutyl.

As used herein, unless otherwise indicated, “C₂₋₄alkenyl” includes but is not limited to 1-propenyl, 2-propenyl, 1-butenyl, 2-butenyl and 3-butenyl.

As used herein, unless otherwise indicated, “C₂₋₄alkynyl” includes but is not limited to ethynyl, 1-propynyl, 2-propynyl, 1-butynyl, 2-butynyl and 3-butynyl.

As used herein, unless otherwise indicated, aryl refers to a phenyl ring which may have 1, 2 or 3 substituents selected from: halogen, C₁₋₄alkyl, C₂₋₄alkenyl, C₂₋₄alkynyl, C₁₋₄alkyl, CN, NO₂, and CF₃.

As used herein, unless otherwise indicated, heteroaryl refers to a 5- or 6-membered aromatic or heteroaromatic ring having 1, 2 or 3 heteroatoms selected from nitrogen oxygen and sulfur, provided that heteroaromatic rings contains at least one nitrogen, oxygen, or sulfur atom.

As used herein, unless otherwise indicated, halogen refers to fluorine, chlorine, bromine, or iodine.

Where necessary, hydroxy, amino, or other reactive groups may be protected using a protecting group as described in the standard text “Protecting groups in Organic Synthesis”, 3^{rd} Edition (1999) by Greene and Wuts.

Unless otherwise stated, reactions are conducted under an inert atmosphere, preferably under a nitrogen atmosphere and are usually conducted at a pressure of about one to about three atmospheres, preferably at ambient pressure (about one atmosphere).

The compounds of the invention and intermediates may be isolated from their reaction mixtures by standard techniques.

Acid addition salts of the compounds of formula I which may be mentioned include salts of mineral acids, for example the hydrochloride and hydrobromide salts; and salts formed with organic acids such as formate, acetate, maleate, benzoate, tartrate, and fumarate salts.

Acid addition salts of compounds of formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

The compounds of formula I exist in tautomeric or enantiomeric forms, all of which are included within the scope of the invention. The various optical isomers may be isolated by separation of a racemic mixture of the compounds using conventional techniques, e.g. fractional crystallisation, or chiral HPLC. Alternatively the individual enantiomers may be made by reaction of the appropriate optically active starting materials under reaction conditions which will not cause racemisation.

Pharmacology

The pharmacological activity of the compounds of the invention may be measured in the tests set out below:

Test A—Assay for Affinity at α₇ nAChR Subtype

¹²⁵I-α-Bungarotoxin (BTX) Binding to Rat Hippocampal Membranes.

Rat hippocampi are homogenized in 20 volumes of cold homogenisation buffer (HB: concentrations of constituents (mM): tris(hydroxymethyl)aminomethane 50; MgCl₂1; NaCl 120; KCl 5: pH 7.4). The homogenate is centrifuged for 5 minutes at 1000×g, the supernatant saved and the pellet re-extracted. The pooled supernatants are centrifuged for 20 minutes at 12000×g, washed, and re-suspended in BB. Membranes (30-80 μg) are incubated with 5 nM [¹²⁵I]α-BTX, 1 mg/mL BSA (bovine serum albumin), test drug, and either 2 mM CaCl₂ or 0.5 mM EGTA [ethylene glycol-bis(β-aminoethylether)] for 2 hours at 21° C., and then filtered and washed 4 times over Whatman glass fiber filters (thickness C) using a Brandel cell harvester. Pre-treating the filters for 3 hours with 1% (BSA/0.01% PEI (polyethyleneimine) in water is critical for low filter blanks (0.07% of total counts per minute). Non-specific binding is described by 100 μM (−)-nicotine, and specific binding is typically 75%.

Test B—Assay for Affinity to the α₄ nAChR Subtype

[³H]-(−)-Nicotine Binding.

Using a procedure modified from Martino-Barrows and Kellar (Mol Pharm (1987) 31:169-174), rat brain (cortex and hippocampus) is homogenised as in the [¹²⁵I]α-BTX binding assay, centrifuged for 20 minutes at 12,000×g, washed twice, and then re-suspended in HB containing 100 μM diisopropyl fluorophosphate. After 20 minutes at 4° C., membranes (approximately 0.5 mg) are incubated with 3 nM [³H]-(−)-nicotine, test drug, 1 μM atropine, and either 2 mM CaCl₂ or 0.5 mM EGTA for 1 hour at 4° C., and then filtered over Whatman glass fiber filters (thickness C) (pre-treated for 1 hour with 0.5% PEI) using a Brandel cell harvester. Non-specific binding is described by 100 μM carbachol, and specific binding is typically 84%.

Binding Data Analysis for Tests A and B

IC50 values and pseudo Hill coefficients (n_{H}) are calculated using the non-linear curve fitting program ALLFIT (DeLean A, Munson P J and Rodbard D (1977) Am. J. Physiol., 235:E97-E102). Saturation curves are fitted to a one site model, using the non-linear regression program ENZFITTER (Leatherbarrow, R. J. (1987)), yielding K_{D} values of 1.67 and 1.70 nM for the ¹²⁵I-α-BTX and [³H]-(−)-nicotine ligands respectively. Kᵢ values are estimated using the general Cheng-Prusoff equation:
Kᵢ=IC₅₀/((2+([ligand]/K_{D})ⁿ)^{1/n}−1)
where a value of n=1 is used whenever n_{H}<1.5 and a value of n=2 is used when n_{H}≦1.5. Samples are assayed in triplicate and were typically ±5%. Kᵢ values are determined using 6 or more drug concentrations. The compounds of the invention are compounds with binding affinities (Kᵢ) of less than 10 μM in either Test A or Test B, indicating that they are expected to have useful therapeutic activity.

The compounds of the invention have the advantage that they may be less toxic, be more efficacious, be longer acting, have a broader range of activity, be more potent, produce fewer side effects, are more easily absorbed or have other useful pharmacological properties.

### Intermediate 1: 1,4-Diazabicyclo[3.2.1]octane

### a) 3-Oxo-piperazin-2-yl-acetic acid ethyl ester

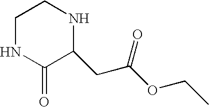

3-Oxo-piperazin-2-yl-acetic acid ethyl ester was prepared according to the procedure described by S. Gubert, et. al. (J. Het. Chem., 30, 1993, 275-276.

### b) 2-piperazin-2-yl-ethanol

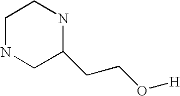

To a mixture of 3-oxo-piperazin-2-yl-acetic acid ethyl ester (2.0 g, 10.74 mmol) in 50 mL of dry THF cooled in an ice bath, was added LAH (1M solution in THF, 20.0 mL, 20.0 mmol) dropwise with stirring under N₂. When addition was complete (c. 10 min), the reaction mixture was refluxed for 3½ h, then cooled in an ice bath. Water (5 mL) was cautiously added with stirring. After stirring for 1 h, the mixture was filtered through a fritted funnel and the collected salts were washed with hot EtOH. The filtrates were combined, dried over MgSO₄, filtered and solvents removed in vacuo. The residue was treated with hot CHCl₃, filtered and the CHCl₃ was evaporated to give a pale yellow oil. The product was obtained in quantitative yield and carried forward without further purification. ¹H NMR (300.132 MHz, CDCl₃) δ 3.82-3.78 (m, 1H), 2.98-2.63 (m, 5H), 2.45-2.36 (m, 1H), 1.62-1.53 (m, 3H), 1.66 (bs, 2H), 1.13 (bs, 1H).

### c) 1,4-Diazabicyclo[3.2.1]octane dihydrochloride salt

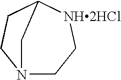

The title compound, 1,4-diazabicyclo[3.2.1]octane was prepared as a dihydrochloride salt from 2-piperazin-2-yl-ethanol according to the procedure described by P. A. Sturn et. al. (J. Med. Chem., 20 (10), 1977, 1333-1337.

### EXAMPLE 1

### 1-(1,4-Diazabicyclo[3.2.1]oct-4-yl)-3-phenyl-propynone

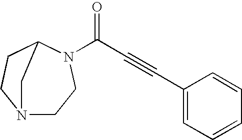

To a stirred solution of phenyl-propynoic acid (32.0 mg, 0.22 mmol), O-(benzotriazol-1-yl)-N,N,N′,N′-tetramethyluronium tetrafluoroborate TBTU (71.0 mg, 0.22 mmol), and 1-hydroxybenzotriazole hydrate (30.0 mg, 0.22 mmol) in DMF (2 mL), was added diisopropylethylamine (0.05 mL, 0.29 mmol). After 5 min, a mixture of 1,4-diazabicyclo[3.2.1]octane dihydrochloride salt (40.0 mg, 0.22 mmol) and 0.1 mL DIEA (0.1 mL, 0.59 mmol) in DMF (1 mL) was added. The reaction mixture was stirred at room temperature overnight. The reaction mixture was then partitioned between EtOAc and 5% Na₂CO₃. The layers were separated and the aqueous phase was extracted with EtOAc. The organic extracts were combined, dried over MgSO₄, filtered and concentrated in vacuo. The residue was chromatographed on silica gel using a gradient of 100:0 to 95:5 CHCl₃:MeOH to afford the title compound as a film. MS (APCI+) 241 [M+1]+. ¹H NMR (300.132 MHz, CDCl₃) δ 7.55 (tt, J=8.3, 1.6 Hz, 2H), 7.44-7.32 (m, 3H), 5.13 (dd, J=5.8, 2.5 Hz, 0.5H), 4.94 (dd, J=5.7, 2.6 Hz, 0.5H), 4.17 (dt, J=8.6, 3.8 Hz, 0.5H), 4.07 (dd, J=13.6, 5.5 Hz, 0.5H), 3.46 (tt, J=13.0, 2.4 Hz, 1H), 3.04 (m, 3H), 2.95 (d, J=12.7 Hz, 1H), 2.83-2.73 (m, 1H), 2.62 (t, J=11.6 Hz, 1H), 2.15-1.83 (m, 2H).

### EXAMPLE 2

### (Z)-1-(1,4-Diazabicyclo[3.2.1]oct-4-yl)-2-fluoro-3-phenyl-propenone

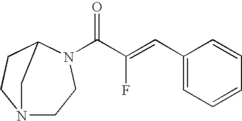

By the process described in Example 1, diazabicyclo[3.2.1]octane dihydrochloride salt was reacted with (Z)-2-fluoro-3-phenyl-acrylic acid to afford the title compound as an off-white solid. MS (APCI+) 261 [M+1]+. ¹H NMR (300.132 MHz, CDCl₃) δ 7.58 (dd, J=7.9, 1.4 Hz, 2H), 7.37 (q, J=7.2 Hz, 3H), 6.60 (d, J=38.6 Hz, 1H), 4.88 (bs, 1H), 3.03 (bs, 1H), 3.39 (bs, 1H), 3.25-3.04 (m, 4H), 2.90 (dd, J=13.5, 4.5 Hz, 1H), 2.78 (d, J=10.8 Hz, 1H), 2.20-1.98 (m, 2H).

### EXAMPLE 3

### (E)-1-(1,4-Diazabicyclo[3.2.1]oct-4-yl)-3-o-tolyl-propenone

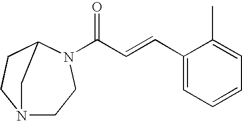

By the process described in Example 1, diazabicyclo[3.2.1]octane dihydrochloride salt was reacted with (E)-3-o-tolyl-acrylic acid to afford the title compound as an off-white solid. MS (APCI+) 257 [M+1]+. ¹H NMR (300.132 MHz, CDCl₃) δ 7.95 (d, J=15.2 Hz, 1H), 7.53 (bs, 1H), 7.23 (d, J=1.7 Hz, 1H), 7.22-7.16 (m, 1H), 6.81 (d, J=14.9 Hz, 1H), 6.65 (d, J=14.9 Hz, 1H), 5.27 (bs, 1H), 4.67-4.17 (m, 1H), 3.80-3.37 (m, 1H), 3.09-2.98 (m, 4H), 2.85-2.74 (m, 1H), 2.65 (d, J=11.4 Hz, 1H), 2.43 (s, 3H), 2.14-1.97 (m, 2H).

### EXAMPLE 4

### 1-(1,4-Diazabicyclo[3.2.1]oct-4-yl)-2-phenoxy-ethanone

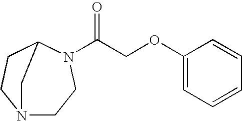

By the process described in Example 1, diazabicyclo[3.2.1]octane dihydrochloride salt was reacted with phenoxy-acetic acid to afford the title compound as a pale yellow solid. MS (APCI+) 247 [M+1]+. ¹H NMR (300.132 MHz, CDCl₃) δ 7.34-7.26 (m, 2H), 6.99 (t, J=7.4 Hz, 1H), 6.98-6.91 (m, 2H), 5.07 (d, J=3.3 Hz, 0.5H), 4.71 (s, 1H), 4.63 (d, J=1.6 Hz, 1H), 4.08 (m, 0.5H), 3.65 (dd, J=5.0, 13.5 Hz, 0.5H), 3.37 (td, J=13.0, 5.0 Hz, 0.5H), 3.02-2.86 (m, 5H), 2.80-2.66 (m, 1H), 2.55 (d, J=11.6 Hz, 1H), 2.05-1.70 (m, 2H).

1. A compound according to Formula I: 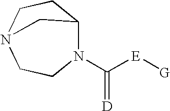
wherein:
D is selected from oxygen, sulfur or N(R¹)₂;
E is selected from —C(R¹)₂—C(R¹)₂—, —CR¹═CR¹—, —C≡C— or —C(R¹)₂—O—;
G is selected from a 5- or 6-membered aromatic or heteroaromatic ring having 0, 1 or 2 nitrogen atoms, 0 or 1 oxygen atoms, and 0 or 1 sulfur atoms, or selected from an 8-, 9- or 10-membered fused aromatic or heteroaromatic ring system having 0, 1, 2 or 3 nitrogen atoms, 0 or 1 oxygen atoms, and 0 or 1 sulfur atoms;
where G is unsubstituted or has 1, 2 or 3 substituents selected from —C₁-C₆alkyl, —C₂-C₆alkenyl, —C₂-C₆alkynyl, halogen, —CN, —NO₂, —CF₃, —S(O)ₙR³, —NR²R³, —CH₂NR²R³, —OR³, —CH₂OR³ or —CO₂R⁴;
R¹, R² and R³ are independently selected at each occurrence from hydrogen, halogen, —C₁-C₄alkyl, aryl, heteroaryl, —C(O)R⁴, —C(O)NHR⁴, —CO₂R⁴ or —SO₂R⁴, or
R² and R³ in combination is —(CH₂)ⱼG(CH₂)ₖ— wherein G is oxygen, sulfur, NR⁴, or a bond;
j is 2, 3 or 4;
k is 0, 1 or 2;
n is 0, 1 or 2, and
R⁴ is independently selected at each occurrence from hydrogen, —C₁-C₄alkyl, aryl, or heteroaryl,
or stereoisomers, enantiomers, in vivo-hydrolysable precursors or a pharmaceutically-acceptable salt thereof.

2. A compound according to claim 1, wherein:
D is oxygen;
E is selected from —C(R¹)₂—C(R¹)₂—, —CR¹═CR¹—, —C≡C— or —C(R¹)₂—O—;
G is phenyl;
where G is unsubstituted or has 1, 2 or 3 substituents selected from —C₁-C₆alkyl and R¹ is independently selected at each occurrence from hydrogen or halogen.

3. A compound according to claim 1, wherein:
D is oxygen;
E is selected from —C(R¹)₂—C(R¹)₂—, —CR¹═CR¹—, —C≡C— or —C(R¹)₂—O—;
G is phenyl;
where G is unsubstituted or has methyl substituent and
R¹ is independently selected at each occurrence from hydrogen or fluoro.

4. A compound according to claim 1, selected from:
1-(1,4-diazabicyclo[3.2.1]oct-4-yl)-3-phenyl-propynone;
(Z)-1-(1,4-diazabicyclo[3.2.1]oct-4-yl)-2-fluoro-3-phenyl-propenone;
(E)-1-(1,4-diazabicyclo[3.2.1]oct-4-yl)-3-o-tolyl-propenone, or
1-(1,4-diazabicyclo[3.2.1]oct-4-yl)-2-phenoxy-ethanone, or a
stereoisomers, enantiomers, in vivo-hydrolysable precursors or a pharmaceutically-acceptable salt thereof.

5. A method of treatment or prophylaxis of a disease or condition in which activation of the α7 nicotinic receptor is beneficial which method comprises administering a therapeutically-effective amount of a compound according to claim 1 to a subject suffering from said disease or condition.

6. The method of claim 5, wherein said disease or condition is anxiety, schizophrenia, mania or manic depression.

7. A method of treatment or prophylaxis of neurological disorders, psychotic disorders or intellectual impairment disorders, which comprises administering a therapeutically effective amount of a compound according to claim 1.

8. The method of claim 7, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, or ulcerative colitis.

9. A method for inducing the cessation of smoking comprising administering an effective amount of a compound according to claim 1.

10. A pharmaceutical composition comprising a compound according to claim 1 and a pharmaceutically-acceptable diluent, lubricant or carrier.

11. A method of treatment or prophylaxis of a disease or condition in which activation of the α7 nicotinic receptor is beneficial which method comprises administering a therapeutically-effective amount of a pharmaceutical composition according to claim 10 to a subject suffering from said disease or condition.

12. The method of claim 11, wherein said disease or condition is anxiety, schizophrenia, mania or manic depression.

13. A method of treatment or prophylaxis of neurological disorders, psychotic disorders or intellectual impairment disorders, which comprises administering a therapeutically effective amount of a pharmaceutical composition according to claim 10.

14. The method of claim 13, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, or ulcerative colitis.

15. A method for inducing the cessation of smoking comprising administering an effective amount of a pharmaceutical composition according to claim 10.

16. The use of a compound according to claim 1, an enantiomer thereof or a pharmaceutically-acceptable salt thereof, in the manufacture of a medicament for the treatment or prophylaxis of human diseases or conditions in which activation of the α7 nicotinic receptor is beneficial selected from neurological disorders, psychotic disorders, intellectual impairment disorders, Alzheimer's disease, learning deficit, cognition deficit, attention deficit, memory loss, Attention Deficit Hyperactivity Disorder, anxiety, schizophrenia, mania or manic depression, Parkinson's disease, Huntington's disease, Tourette's syndrome, or neurodegenerative disorders in which there is loss of cholinergic synapses.

17. The use of a compound according to claim 1, in the manufacture of a medicament for the treatment or prophylaxis of jetlag, pain, or ulcerative colitis or to facilitate the cessation of smoking or the treatment of nicotine addiction or craving including that resulting from exposure to products containing nicotine.
